(19) United States
(12) Patent Application Publication
(10) Pub. No.: US 2026/0198817 A1
(43) Pub. Date: Jul. 16, 2026

(54) SAMPLING APPARATUS, SAMPLER, AND SAMPLING METHOD

(71) Applicant: SUZHOU NATONG BIOMED CO., LTD, Suzhou, Jiangsu (CN)

(72) Inventor: Bai Xu, Suzhou, Jiangsu (CN)

(21) Appl. No.: 19/134,261

(22) PCT Filed: Nov. 2, 2023
(86) PCT No.: PCT/CN2023/129264

§ 371 Date: May 30, 2025

(30) Foreign Application Priority Data
Dec. 2, 2022 (CN) 202211536269.2

(51) Int. Cl. A61B 5/15

(52) U.S. Cl. CPC A61B 5/150099; A61B 5/150412; A61B 5/150984

(57) ABSTRACT

Provided are a sampling apparatus, a sampler, and a sampling method. The sampling apparatus comprises a housing, provided with a sampling window capable of making contact with skin; a negative pressure unit, which can reduce the air pressure inside the housing and form an air pressure difference at the sampling window so as to adhere the skin; and a driving unit, comprising a driver and a transmission rod. An assembly part is formed at an end of the transmission rod, and the driver can control the assembly part to reciprocate towards the sampling window by driving the transmission rod to axially reciprocate.

### TECHNICAL FIELD

The present disclosure relates to the technical field of microneedle sampling, and particularly, to a sampling apparatus, a sampler, and a sampling method.

### BACKGROUND ART

In clinical diagnosis and treatment, body fluid sampling and analysis are important methods for assessing a patient's health condition. Body fluids include tissue fluid, blood, lymph, etc. Medical staff can perform convenient and efficient diagnostic tests by obtaining a small amount of body fluid from the patient's subcutaneous tissue or capillaries. Currently, the mainstream sampling method involves puncturing the fingertip with a steel needle and then by the way of squeezing the finger to collect an adequate amount of body fluid for clinical diagnosis. This method has many drawbacks: firstly, the sampling process causes a relatively great pain, especially psychological trauma on children, leading to obvious resistance to psychology; at the same time, after sampling, a cotton ball is required to apply pressure to stop the bleeding, which is inconvenient for the patient; and additionally, the wound takes a long time to heal, and improper handling can easily lead to infections and other issues.

For this reason, a more convenient sampling device is disclosed in the prior art, such as the technical solution provided in the Chinese invention patent application “INTEGRATED BLOOD GLUCOSE COLLECTION AND DETECTION PEN (patent application number: CN201711412980.6)”, the steel needle is controlled to pop out quickly through a spring and a striking mechanism, and the needle insertion depth can be adjusted according to individual differences of patients, so as to reduce the pain during sampling. This improved solution can reduce the discomfort to a certain extent during sampling, but the selection of needle insertion depth requires a relatively high level of operator experience. If the needle is inserted too deeply, it may increase the pain; if it is inserted too shallowly, body fluid cannot flow out, resulting in the failure of blood sampling. In addition, the internal structure of this device is complex, and the spring needs to be pre-compressed or pre-stretched during assembly, which requires high assembly requirements. Moreover, this technology still fails to address the issue of wound infection after sampling. Furthermore, for the diseases requiring long-term monitoring such as diabetes, many patients opt for at-home self-sampling and monitoring. For these patients, the operational difficulty of such devices becomes even greater. Therefore, there remain areas for improvement in the existing technical solutions in the field of body fluid sampling.

### SUMMARY

### Technical Issues

In order to solve the technical issues existing in traditional body fluid sampling, such as sampling pain, difficult operation, inconvenient wound care, susceptibility to wound infection, and complicated equipment etc., a sampling apparatus is provided in the present disclosure.

### Technical Solutions

The sampling apparatus includes a housing, wherein a sampling window capable of contacting with skin is arranged on the housing; a negative pressure unit, wherein the negative pressure unit can reduce the air pressure inside the housing and form an air pressure difference at the sampling window, so as to adhere the skin; and a driving unit, wherein the driving unit includes a driver and a transmission rod. An assembly part is formed at the end of the transmission rod, and the driver can control the assembly part to reciprocate towards the sampling window by driving the transmission rod to axially reciprocate.

The relevant contents of the above technical solutions are further explained as follows.

The negative pressure unit aspirates the gas inside the housing, causing the air pressure in the internal space of the housing to decrease. Relative to the external air pressure outside the housing, the interior of the housing becomes a low-pressure zone. Since the internal space of the housing is connected to the external environment through the sampling window, an air pressure difference is formed at the sampling window. When the sampling window is aligned and in contact with the skin, the sampling window can adhere to the skin under the action of air pressure difference. According to the present disclosure, the acting force that the sampling window adheres to the skin due to the “air pressure difference” is defined as the “specific suction force”. The specific suction force can stabilize the skin and facilitate the extraction of body fluid. The magnitude of the specific suction force is related to the magnitude of the “air pressure difference” and can be adjusted by the power of the negative pressure unit.

According to the present disclosure, the target skin area to be sampled can be accurately captured through the specific suction force, thus ensuring the accuracy of the sampling position. The assembly part on the driving unit is designed to match with various sampling units, and various body fluids can be sampled by assembling different sampling units. During sampling, the assembly part on the driving unit can move reciprocally toward the sampling window, thereby controlling the sampling unit assembled thereon to reciprocally contact or puncture the target skin area to be sampled, thus completing the sampling and treatment of the skin.

The sampling apparatus in the present disclosure has a novel structure and a unique working principle. By using a driver to drive the sampling unit to perform multiple times of reciprocal movements with high frequency in a short time instead of the traditional one-time spring striking, a plurality of sampling channels can be instantly formed on the skin surface. This improvement allows the sampling unit to be equipped with a finer needle type or other types, effectively reducing puncture pain while maintaining sampling efficiency. Thinner needle type can not only reduce the pain during puncture, but also produce smaller wounds. Under the action of skin viscoelasticity and the self-repair capability of blood cells, tiny wounds can heal faster, thus further reducing the risk of wound infection. In addition, the sampling apparatus of the present disclosure can accurately locate the sampling position during the use process, making the operation precise, simple, and convenient. In the sampling process, the specific suction force generated by the negative pressure unit can promote the outflow of body fluids, eliminating the need to squeeze muscle tissues such as fingers during sampling, thus further reducing pain and avoiding skin trauma, and alleviating the resistance and fear of patients.

In the preferred technical solution of the present disclosure, a partition part is arranged in the housing, wherein the partition part divides the interior of the housing into at least two chambers, including a first chamber and a second chamber, wherein the first chamber is communicated with the sampling window. The internal space of the housing is divided into a plurality of chambers by arranging partition parts. Through the above configuration, all components can be arranged more firmly and regularly, thus improving the structural stability. A separate chamber can be formed by the partition part, and the volume of the chamber can be changed by adjusting the position of the partition part. By reducing the volume of the first chamber, the negative pressure unit can generate enough specific suction force in the sampling window with less power, thus reducing the power requirements for the negative pressure unit, so as to reduce energy consumption. In addition, the independent second chamber can better protect all electrical components and avoid the mixing or crossing of bacteria and dust in the two chambers.

In the preferred technical solution of the present disclosure, the driving unit is arranged in the first chamber. Through the above configuration, the driving unit is integrally arranged in the first chamber, thereby simplifying the structure effectively and reducing the assembly difficulty.

In the preferred technical solution of the present disclosure, the driver is arranged in the second chamber, the transmission rod penetrates through the partition part, and the assembly part extends into the first chamber. Through the above configuration, the volume of the first chamber can be further reduced, thereby reducing the power requirement for the negative pressure unit.

In the preferred technical solution of the present disclosure, a sliding sealing ring is arranged between the transmission rod and the partition part, so that the transmission rod keeps an air-tight state with the partition part during the axial reciprocating movements relative to the partition part.

In the preferred technical solution of the present disclosure, the negative pressure unit includes an aspiration pump and an aspiration tube, wherein one end of the aspiration tube is connected to the air inlet of the aspiration pump, and the other end of the aspiration tube is communicated with the sampling window. The aspiration pump can achieve automatic aspiration, thus further improving operational convenience.

In the preferred technical solution of the present disclosure, the negative pressure unit reduces the air pressure in the housing by aspirating gas, and the air pressure difference formed at the sampling window is not less than 1 MPa.

A sampler is further provided in the present disclosure, wherein the sampler includes the sampling apparatus according to any one of the above technical solutions, and the sampling unit. The sampling unit is detachably assembled on the sampling apparatus and can be driven by the sampling apparatus to puncture the skin in a reciprocating way at the sampling window.

In the present technical solution, the sampling apparatus can drive the sampling unit to reciprocally puncture the skin multiple times at high frequency, so that a plurality of sampling channels can be formed on the skin surface in a short time, thus improving the exudation speed of body fluids. Based on this improvement, a sampling unit with a finer size can be adopted by the sampler in the present disclosure, thereby reducing the pain of patients and at the same time reducing the wound area. Since the wound area is reduced, its recovery period is shorter, so that the risk of wound infection can be further reduced. Therefore, the present disclosure has multi-directional technological advancement.

In the preferred technical solution of the present disclosure, the sampling unit includes one or more needle bodies. The needle body can be a steel needle, a silicon needle, or other suitable needle body.

In the preferred technical solution of the present disclosure, the sampling unit includes a base plate and micro-nano needles arranged in an array on the surface of the base plate. It should be noted that the micro-nano needle includes a needle type with a needle body length at the micrometer scale and a needle tip size at the nanometer scale. A relatively mature product in the prior art is the nano-wafer. By configuring micro-nano needles, multiple sampling channels can be formed on the skin surface, effectively improving the sampling efficiency; at the same time, the nano-scale sampling channel will not produce obvious pain, effectively reducing the user's discomfort.

In the preferred technical solution of the present disclosure, the micro-nano needle is a solid needle type. Through the above configuration, the body fluid flows out during the time interval when the micro-nano needle reciprocally punctures the skin. The solid needle type is easier to manufacture, with good stability and low cost, so that the sampling costs can be effectively reduced.

In the preferred technical solution of the present disclosure, the spacing between any adjacent micro-nano needles is not less than 0.1 mm.

A sampling method is further provided in the present disclosure, wherein the sampling method includes the following steps:
disinfecting and cleaning the skin surface;
performing sampling treatment on the skin surface by using the sampler described in any one of the above technical solutions; and
adopting a test paper to absorb body fluids exuded from the skin surface.

In the preferred technical solution of the present disclosure, the sampling unit of the sampler includes a base plate and micro-nano needles arranged in an array on the surface of the base plate, wherein the micro-nano needles are in solid needle type, and the spacing between any adjacent micro-nano needles is not less than 0.1 mm.

In the preferred technical solution of the present disclosure, the step of performing sampling treatment on the skin surface by using the sampler described in any one of the above technical solutions includes:
making the sampling window face directly and in contact with the skin surface;
starting a negative pressure unit, wherein the negative pressure unit generates a specific suction force at the sampling window and the skin is adhered;
starting a driving unit, wherein the driving unit controls the sampling unit to perform reciprocating puncture on the skin surface;
turning off the driving unit, and keeping the negative pressure unit working for a specific duration;
turning off the negative pressure unit; and
removing the sampler.

It is worth emphasizing that after the driving unit controls the sampling unit to puncture the skin surface, the negative pressure unit keeps working for a specific duration, so that the outflow of body fluids can be effectively promoted, thus improving the sampling efficiency.

### The Beneficial Effects

The sampling apparatus is simple and exquisite in structure, and can be matched with different sampling units to form a sampler. The sampler controls the sampling units to repeatedly puncture the skin in a short time with high frequency to form a plurality of fine sampling channels, and moreover, obvious pain will not be produced by these sampling channels. The sampler can adopt a needle type with a finer size, thus forming a small wound while ensuring the sampling efficiency, so that the infection probability can be effectively reduced.

### BRIEF DESCRIPTION OF THE DRAWINGS

Preferred embodiments of the present disclosure will be described with reference to the accompanying drawings, in which:

FIG. 1 is a schematic structural diagram of the sampling apparatus according to the example of the present disclosure.

FIG. 2 is a schematic structural diagram of the sampler according to Example 1 of the present disclosure.

FIG. 3 is an enlarged schematic diagram at T in FIG. 2.

FIG. 4 is a schematic structural diagram of the sampler according to Example 2 of the present disclosure.

FIG. 5 is a flowchart of a sampling method according to the present disclosure.

FIG. 6 is a detailed step diagram of the sampling method according to the present disclosure.

### LIST OF REFERENCE NUMERALS

A. sampling apparatus; B. sampler; Q1. first chamber; Q2. second chamber; 1. housing; 10. sampling head; 11. sampling window; 12. exhaust hole; 13. partition part; 131. sliding sealing ring; 2. negative pressure unit; 21. aspiration pump; 22. aspiration tube; 221. aspiration end; 23. exhaust end; 3. driving unit; 31. driver; 32. transmission rod; 321. assembly part; 4. sampling unit; 41. mounting base; 42. base plate; 43. micro-nano needle; 5. power supply.

### DETAILED DESCRIPTION OF EMBODIMENTS

Preferred embodiments of the present disclosure will be described below with reference to the accompanying drawings. It should be understood by those skilled in the art that these embodiments are only used to explain the technical principles of the present disclosure, and are not intended to limit the protection scope of the present disclosure.

It should be noted that in the description of the present disclosure, the terms of direction or positional relationship indicated by the terms “up”, “down”, “inside” and “outside”, and the like indicate directions or positional relationships based on those shown in the accompanying drawings, which is only for the convenience of description, and does not indicate or imply that the device or element must have a specific orientation, be constructed and operated in a specific orientation, and therefore are not to be understood as limitations of the present disclosure. Furthermore, the terms “first” and “second” are only used for descriptive purposes and cannot be understood as indicating or implying relative importance.

In addition, it should be noted that in the description of the present disclosure, unless otherwise specified and limited, the terms “assembly”, “provide” and “connect” should be broadly understood, for example, it can be fixed connection, detachable connection or integrated connection; it can be directly connected, or be indirectly connected through an intermediate medium, and further can be communication inside two elements. For those skilled in the art, the specific meanings of the above terms in the present disclosure can be understood according to specific circumstances.

In order to solve the technical issues existing in traditional body fluid sampling, such as sampling pain, inconvenient operation, susceptibility to wound infection, unstable sampling success rate, complicated equipment, and complicated production and assembly, etc., a sampling apparatus A is provided in the present disclosure. The sampling apparatus A includes a housing 1, wherein a sampling window 11 capable of contacting with skin is arranged on the housing 1; a negative pressure unit 2, wherein the negative pressure unit 2 can reduce the air pressure inside the housing 1 and form an air pressure difference at the sampling window 11, so as to adhere the skin; and a driving unit 3, wherein the driving unit 3 includes a driver 31 and a transmission rod 32. An assembly part 321 is formed at the end of the transmission rod 32, and the driver 31 can control the assembly part 321 to reciprocate towards the sampling window 11 by driving the transmission rod 32 to axially reciprocate.

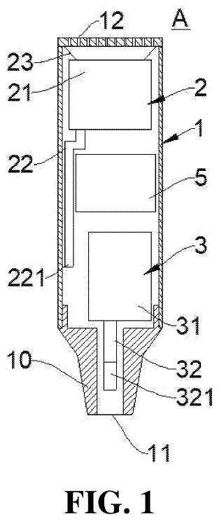

FIG. 1 is a schematic structural diagram of the sampling apparatus according to the example of the present disclosure. As shown in FIG. 1, in one example, the housing 1 is in a straight cylinder shape, the upper end of the housing 1 is provided with an exhaust hole 12, and the lower end of the housing 1 includes a detachable sampling head 10, wherein the sampling window 11 is opened on the sampling head 10. It is readily appreciated that the sampling head 10 can further be formed in an integrally molded design with the housing 1, or the sampling window 11 can be formed directly at the lower end of the housing 1. A negative pressure unit 2, a driving unit 3 and a power supply 5 are arranged inside the housing. As shown in FIG. 1, the negative pressure unit 2 includes an aspiration pump 21, an aspiration tube 22 and an exhaust end 23, wherein the aspiration end 221 of the aspiration tube 22 is arranged in the cavity of the housing 1, and the exhaust end 23 is communicated with the exhaust hole 12. When the aspiration pump 21 works, the gas in the cavity of the housing 1 is aspirated by the aspiration end 221 and discharged from the exhaust end 23 through the exhaust hole 12, so that the air pressure in the space of the housing 1 is reduced and a specific suction force is generated at the position of the sampling window 11.

As shown in FIG. 1, the driving unit 3 includes a driver 31, wherein the power output end of the driver 31 is connected with the transmission rod 32, and an assembly part 321 is arranged at the end of the transmission rod 32. In one or more examples, the assembly part 321 and a part of the transmission rod 32 extend into the sampling head 10 and are close to the sampling window 11. When the driver 31 drives the transmission rod 32 to reciprocate in the axial direction, the assembly part 321 can reciprocate toward the sampling window 11. As shown in FIG. 1, the negative pressure unit 2 and the driving unit 3 are powered by a power supply 5.

The sampling apparatus A in the present disclosure can be used with different types of sampling units, so as to sample different types of body fluids for various diagnostic treatments. In the following, further explanation is provided by some examples.

### Example 1

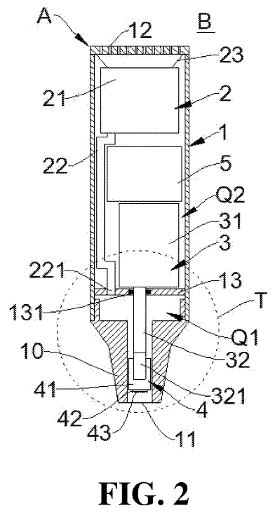

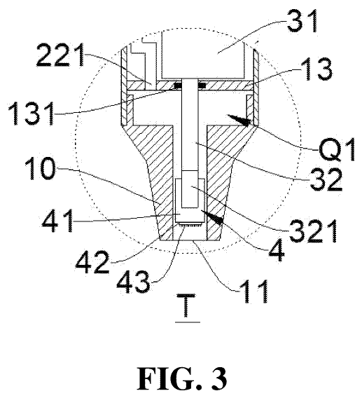

FIG. 2 is a schematic structural diagram of the sampler according to Example 1 of the present disclosure, and FIG. 3 is an enlarged schematic diagram at T in FIG. 2. In the present Example 1, the sampler B is formed by combining the sampling apparatus A with a sampling unit 4.

As shown in FIG. 2 and FIG. 3, the housing 1 of the sampling apparatus A is in a straight cylinder shape. Alternatively, the housing 1 can be L-shaped, T-shaped, square or other suitable shapes. The upper end of the housing 1 is provided with a plurality of exhaust holes 12 arranged in an array. Alternatively, only one exhaust hole 12 can be provided. The shape of the exhaust hole 12 can be a round hole, a square hole, a hexagonal hole or other suitable shapes. It is readily contemplated that the exhaust hole 12 can further be opened on a side perimeter wall of the housing or other suitable positions. As shown in FIG. 2, a sampling head 10 is detachably arranged at the lower end of the housing 1, wherein the sampling head 10 can be assembled on the housing 1 by means of snap fit, thread, plug-in connection, etc. Alternatively, the sampling head 10 and the housing 1 are designed as an integrally formed structure. As shown in FIG. 2 and FIG. 3, the sampling head 10 is in the shape of a downwardly tapered frustum, and a hollow channel is formed in the sampling head 10. The upper end of the hollow channel is communicated with the internal space of the housing, and the lower end of the hollow channel forms the sampling window 11. It is readily understandable that in some examples, the lower end of the housing 1 can directly form the sampling window 11, and optionally, it can be assembled with a specific sampling unit 4 to form an air-tight sealed connection, so as to facilitate the generation of specific suction force at the sampling window 11.

As shown in FIG. 2, a partition part 13 is provided on the housing 1. The partition part 13 divides the internal space of the housing 1 into a first chamber Q1 and a second chamber Q2, wherein the first chamber Q1 communicates with the sampling window 11 through a hollow channel on the sampling head 10. It is readily contemplated that the partition part 13 can further divide the internal space of the housing into more chambers, thus facilitating the installation and arrangement of various electrical components.

As shown in FIG. 2 and FIG. 3, the negative pressure unit 2 is arranged in the second chamber Q2. In the present Example 1, the negative pressure unit 2 realizes gas aspiration by the aspiration pump 21 and generates a specific suction force. Alternatively, the negative pressure unit 2 can further include a piston cylinder, a piston disc, or other suitable manual or electric means for gas aspiration. As shown in FIG. 2, the aspiration pump 21 is connected to an aspiration tube 22, wherein the end of the aspiration tube 22 is fixed on the partition part 13 and forms an aspiration end 221. The negative pressure unit 2 communicates with the first chamber Q1 through the aspiration tube and the aspiration end. As shown in FIG. 2, the exhaust end 23 of the aspiration pump 21 is in a bowl shape and communicates with the exhaust hole 12. It is readily contemplated that the exhaust end 23 can further be tubular or other suitable structures.

As shown in FIG. 2, in the present Example 1, the driver 31 of the driving unit 3 is arranged in the second chamber Q2. The driver 31 can be an eccentric motor with a transmission structure and can further be a linear motor, an electromagnetic vibrator or other suitable driving components. A driving rod 32 is connected to the power output end of the driver 31. As shown in FIG. 2, in the present Example 1, the driving rod 32 penetrates the partition part 13 and then extends into the first chamber Q1. The end of the driving rod 32 is provided with an assembly part 321, wherein the assembly part 321 extends close to the sampling window 11. The assembly part 321 can be designed to be integrally formed with the driving rod 32 in the shape of a plug-in shaft, or can take the form of a socket, plug-in cylinder, or other suitable assembly structure. It is readily understandable that the assembly part 321 can be fixed on the driving rod 32 by welding, threaded connection, snap-fit connection, etc.

As shown in FIG. 2 and FIG. 3, in order to further ensure the specific suction force generated by the negative pressure unit 2 at the sampling window 11, in the present Example 1, a sliding sealing ring 131 is provided between the driving rod 32 and the partition part 13. The sliding sealing ring 131 can be a resilient rubber member, a sealing washer, a sealing bearing, or other suitable sealing component. In addition, a sealing lubricant can be provided between the driving rod 32, the sliding sealing ring 131 and the partition part 13, so as to reduce the wear between them.

As shown in FIG. 2, in the present Example 1, the power supply 5 is arranged in the second chamber Q2 for supplying electric energy to the aspiration pump 21 and the driver 31. The power supply 5 can be a detachable and replaceable dry battery, or can be a rechargeable battery that does not need to be replaced. If the power supply 5 is a rechargeable battery, the charging port can be arranged at the upper end of the housing 1, or the side perimeter wall of the housing 1, or other suitable positions. In addition, the power supply 5 can be replaced by a wired power supply, wherein the power cord extends from the upper end or the side perimeter wall of the housing 1 to the outside of the housing 1.

As shown in FIG. 2 and FIG. 3, the sampling unit 4 is detachably mounted on the assembly part 321. In the present Example 1, the sampling unit 4 includes a mounting base 41, wherein the mounting base 41 is sleeved over the assembly part 321. The specific fixing method of the mounting base 41 and the mounting part 321 can be threaded screw connection, snap-fit connection, interference fit, or other suitable connection methods. It is readily understandable that when the configuration of the assembly head 321 is changed, the mounting base 41 can be adaptively modified and achieve stable assembly with it. As shown in FIG. 2, the sampling unit 4 further includes a nano-wafer, wherein the nano-wafer includes a base plate 42 and micro-nano needles 43 arranged on the surface of the base plate 42. The base plate 42 is fixed on the mounting base 41, and the micro-nano needle 43 faces the sampling window 11. In the present example, the micro-nano needle 43 is a solid needle type, wherein the height of the needle body is in the micrometer scale, the needle tip size is in the nanometer scale, and the spacing between any adjacent micro-nano needles 43 is not less than 0.1 mm.

### Embodiments of the Present Disclosure

### Example 2

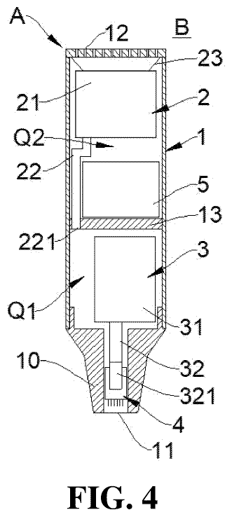

FIG. 4 is a schematic structural diagram of the sampler according to Example 2 of the present disclosure. Different from Example 1, in the present Example 2, the driving unit 3 is integrally arranged in the first chamber Q1, and the driver 31 is electrically connected with the power supply 5 through wires. In addition, in the present Example 2, the sampling unit 4 includes a mounting base and a plurality of needle bodies, wherein the needle bodies are fixed on the mounting base in parallel with each other, and the needle tips face the sampling window 11. Alternatively, only one needle body is arranged on the sampling unit 4. The needle body can be a steel needle, a silicon needle or other suitable needle body.

The use method and details of sampler B are further explained below.

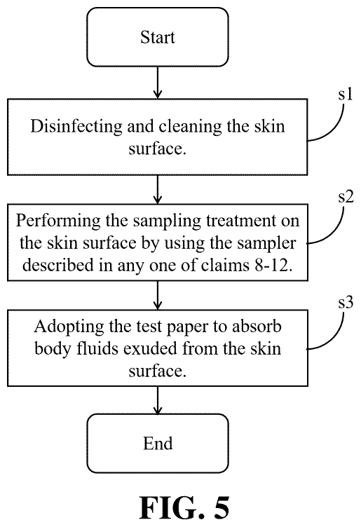

FIG. 5 is a flowchart of a sampling method according to the present disclosure. As shown in FIG. 5, the sampling method includes the following steps:
disinfecting and cleaning the skin surface (step s1);
performing sampling treatment on the skin surface by using the sampler B (step s2); and
using a test paper to absorb body fluids exuded from the skin surface (step s3).

In one or more sampling scenarios, the sampling unit 4 of the sampler B includes a base plate 42 and micro-nano needles 43 arranged in an array on the surface of the base plate 42, wherein the micro-nano needles 43 are in solid needle type, and the spacing between any adjacent micro-nano needles 43 is not less than 0.1 mm.

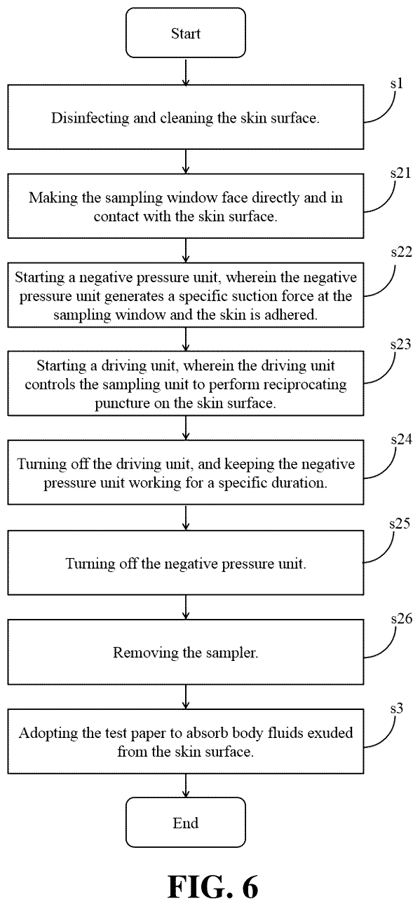

FIG. 6 is a detailed step diagram of the sampling method according to the present disclosure. As shown in FIG. 6, the sampling method specifically includes the following steps:
disinfecting and cleaning the skin surface (step s1);
making the sampling window 11 face directly and in contact with the skin surface (step s21);
starting the negative pressure unit 2, wherein the negative pressure unit 2 generates a specific suction force at the sampling window 11 and the skin is adhered (step s22);
starting the driving unit 3, wherein the driving unit 3 controls the sampling unit 4 to perform reciprocating puncture on the skin surface (step s23);
turning off the driving unit 3, and keeping the negative pressure unit 2 working for a specific duration (step s24);
turning off the negative pressure unit 2 (step s25);
removing the sampler B (step s26); and
using a test paper to absorb body fluids exuded from the skin surface (step s3).

Some operational details are further explained below.

In step s1, alcohol with a concentration of 75% can be selected to wipe the skin surface to disinfect and clean the skin.

After the negative pressure unit 2 is started, the aspiration pump 21 continuously aspirates the gas in the first chamber Q1, making the air pressure in the first chamber Q1 decrease, so that an air pressure difference is generated at the sampling window 11, wherein the air pressure difference creates a specific suction force to aspirate the skin into the sampling window 11, causing a certain degree of arching of the skin surface. In step s22, by changing the power of the negative pressure unit 2, the air pressure difference at the sampling window 11 can be adjusted, and then the magnitude of specific suction force can be adjusted, thereby adjusting the suction force of the sampler B on the skin. Alternatively, the negative pressure unit 2 makes the air pressure difference generated at the sampling window 11 not less than 1 MPa by aspirating gas. Preferably, the air pressure difference is 5 MPa, 15 MPa, or 20 MPa.

When the skin surface is stably adhered by the sampling window 11, step s23 is executed. The driving unit 3 controls the sampling unit 4 to reciprocally puncture the skin surface with high frequency in a short time, thus forming a plurality of sampling channels on the skin surface instantly. In the present embodiment, the height of the micro-nano needle body is in the micrometer scale, and the size of the needle tip is in the nanometer scale, so that the formed wound is fine, thereby effectively relieving the pain during sampling. At the same time, a large number of sampling channels generated in a short time can ensure the efficient exudation of body fluids.

After the sampling treatment of the skin by the sampling unit 4 is completed, the negative pressure unit 2 is continuously maintained in working, and the low air pressure in the first chamber Q1 can promote the exudation of body fluids, thus improving the sampling efficiency.

It should be noted that since the sampler B in the present disclosure can form a plurality of sampling channels on the skin surface in a short time, when a plurality of needles or a single needle is selected by the sampling unit 4, the sampler B can quickly obtain enough body fluid through a specific suction force even if the needle size is fine. Moreover, when the needle body with a fine size is selected, the pain of patients can be relieved obviously, and at the same time, the size of the wound can be reduced, thus shortening the recovery time of the wound and reducing the probability of infection.

So far, the technical solution of the present disclosure has been described in combination with the preferred embodiments shown in the accompanying drawings, but it is easy for those skilled in the art to understand that the protection scope of the present disclosure is obviously not limited to these specific embodiments. Without deviating from the principle of the present disclosure, those skilled in the art can make equivalent changes or substitutions on relevant technical features, and the technical solutions after these changes or substitutions will fall within the protection scope of the present disclosure.

1. A sampling apparatus, wherein the sampling apparatus comprises:
a housing, wherein a sampling window capable of contacting with a skin is arranged on the housing;
a negative pressure unit, wherein the negative pressure unit can reduce an air pressure inside the housing and form an air pressure difference at the sampling window, so as to adhere the skin; and
a driving unit, wherein the driving unit comprises a driver and a transmission rod;
an assembly part is formed at an end of the transmission rod, and the driver can control the assembly part to reciprocate towards the sampling window by driving the transmission rod to axially reciprocate.

2. The sampling apparatus according to claim 1, wherein a partition part is arranged in the housing, the partition part divides an interior of the housing into at least two chambers, wherein a first chamber is communicated with the sampling window.

3. The sampling apparatus according to claim 2, wherein the driving unit is arranged in the first chamber.

4. The sampling apparatus according to claim 2, wherein the driver is arranged in a second chamber, the transmission rod penetrates through the partition part, and the assembly part extends into the first chamber.

5. The sampling apparatus according to claim 4, wherein a sliding sealing ring is arranged between the transmission rod and the partition part, so that the transmission rod keeps an air-tight state with the partition part during an axial reciprocating movement relative to the partition part.

6. The sampling apparatus according to claim 1, wherein the negative pressure unit comprises an aspiration pump and an aspiration tube, wherein one end of the aspiration tube is connected to an air inlet of the aspiration pump, and the other end of the aspiration tube is communicated with the sampling window.

7. The sampling apparatus according to claim 6, wherein the negative pressure unit reduces the air pressure in the housing by aspirating gas, and the air pressure difference formed at the sampling window is not less than 1 MPa.

8. A sampler, wherein the sampler comprises:
the sampling apparatus according to claim 1; and
a sampling unit, wherein the sampling unit is detachably assembled on the sampling apparatus and can be driven by the sampling apparatus to puncture the skin in a reciprocating way at the sampling window.

9. The sampler according to claim 8, wherein the sampling unit comprises one or more needle bodies.

10. The sampler according to claim 8, wherein the sampling unit comprises a base plate and micro-nano needles arranged in an array on a surface of the base plate.

11. The sampler according to claim 10, wherein the micro-nano needle is in a solid needle type.

12. The sampler according to claim 11, wherein a spacing between any adjacent micro-nano needles is not less than 0.1 mm.

13. A sampling method, wherein the sampling method comprises following steps:
disinfecting and cleaning a skin surface;
performing a sampling treatment on the skin surface by using the sampler according to claim 8; and
adopting a test paper to absorb body fluids exuded from the skin surface.

14. The sampling method according to claim 13, wherein a sampling unit of the sampler comprises a base plate and micro-nano needles arranged in the array on the surface of the base plate; and the micro-nano needles are in a solid needle type, and a spacing between any adjacent micro-nano needles is not less than 0.1 mm.

15. The sampling method according to claim 14, wherein the step of performing sampling treatment on the skin surface by using the sampler comprises:
making the sampling window face directly and in contact with the skin surface;
starting a negative pressure unit, wherein the negative pressure unit generates a specific suction force at the sampling window and the skin is adhered;
starting a driving unit, wherein the driving unit controls the sampling unit to perform reciprocating puncture on the skin surface;
turning off the driving unit, and keeping the negative pressure unit working for a specific duration;
turning off the negative pressure unit; and
removing the sampler.
